SEQUENCE LISTING (17 sequences)

SEQ ID NO: 1 - DNA, 29 bp, Artificial sequence
  Synthesized
ggagagtctagaatgagcacaaaaccttt

SEQ ID NO: 2 - DNA, 31 bp, Artificial sequence
  Synthesized
cgctgcgtcgacttaattgcttatatttacc

SEQ ID NO: 3 - DNA, 20 bp, Artificial sequence
  Synthesized
cagctcatcaaccaggtcaa

SEQ ID NO: 4 - DNA, 20 bp, Artificial sequence
  Synthesized
aaaagccgtcacgttattgg

SEQ ID NO: 5 - DNA, 20 bp, Artificial sequence
  Synthesized
agcgttatctcgcggaccgt

SEQ ID NO: 6 - DNA, 20 bp, Artificial sequence
  Synthesized
gcatttgtttgcagtgatcg

SEQ ID NO: 7 - DNA, 20 bp, Artificial sequence
  Synthesized
ggagaaaataccgcatcagg

SEQ ID NO: 8 - DNA, 18 bp, Artificial sequence
  Synthesized
gcgttggccgattcatta

SEQ ID NO: 9 - DNA, 20 bp, Artificial sequence
  Synthesized
ggcatgcatttacgttgaca

SEQ ID NO: 10 - DNA, 20 bp, Artificial sequence
  Synthesized
agaaacgcaaaaaggccatc

SEQ ID NO: 11 - DNA, 70 bp, Artificial sequence
  Synthesized
atggaactgacgactcgcactttacctgcgcggaaacatattgcgctggtgtgtaggctggagctgcttc

SEQ ID NO: 12 - DNA, 65 bp, Artificial sequence
  Synthesized
gacaagtcaatttccgcccacgtatctctgtaaaaaggttttgtgctcatagctgtttcctggtt

SEQ ID NO: 13 - DNA, 20 bp, Artificial sequence
  Synthesized
gcagcgattgccacatactc

SEQ ID NO: 14 - DNA, 1170 bp, Bacillus subtilis
  Organism
atgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaa

SEQ ID NO: 15 - DNA, 1435 bp, Escherichia coli
  Organism
cagctcatcaaccaggtcaattttcgcctgcctcgcctggagaatgagcgggaagctaaccattttgatgccgcgccgcaccttgaattgttggatggcgatgcgacgccaattccggtaaagctggatattctcgccattacctcaacggcaaaaacgattgattatgaggtcgaggttgaacgctataacaaatccgcgaaacgcgcttcactaccgcaatttgccacgatgatggcagatgacagtacgctgctttcgggtgtttccgagctggatgctattccaccgcagtctcaggtgctcacagaacaacggctgaagtattggtttaaactggctgacccacaaacgcgaaatactttcctgcagtgggcggaaaaacaaccatcttcctgagatttttgtgcctgtgcgcaggctttttcggtctttatcttgcagcgataagtgcttacagtaatctgtaggaaagttaactacggatgtacattatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccaccggcacatccagatttttcagtatttccacaaagagagagggattctctttgttaaacagtgcccatacccgatggcgtgcccgggtcagcgccacgtacattaaccgccgttcttcagcgtccgggaaatcctcaaccggtggcagtagcgcctcttccataatcgactcccgcgccgcagccggaaaaccatcacttccctcctgcaagccaacgatgatgacgtaatccgcctgttgccctttgctggcatgaatggtcataaagtcgatttgcaacttcggccagcgtgttgccgctttttccaggctggcaggcctcatgtgatggtaacgcgccaggatcagaatgcgctcttccggtttggcataaccagagagcttatccagcaaagcgtcaagttgactctcatccaataacgtgacggctttt

SEQ ID NO: 16 - DNA, 1664 bp, Artificial sequence
  Synthesized
atggaactgacgactcgcactttacctgcgcggaaacatattgcgctggtgtgtaggctggagctgcttcgaagttcctatactttctagagaataggaacttcggaataggaacttcaagatcccccacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatgttatctctggcggtgttgacaagagataacaacgttgatataattgagcccgtattgttagcatgtacgtttaaaccaggaaacagctatgagcacaaaacctttttacagagatacgtgggcggaaattgacttgtc

SEQ ID NO: 17 - DNA, 88 bp, Artificial sequence
  Synthesized
ttatctctggcggtgttgacaagagataacaacgttgatataattgagcccgtattgttagcatgtacgtttaaaccaggaaacagct

(19) United States
(12) Patent Application Publication
(10) Pub. No.: US 2015/0247174 A1
(43) Pub. Date: Sep. 3, 2015

(54) ENGINEERING BACTERIA FOR PRODUCING DL-ALANINE AND METHOD FOR PRODUCING DL-ALANINE BY USING ENGINEERING BACTERIA

(71) Applicant: ANHUI HUAHENG BIOENGINEERING CO., LTD., Hefei (CN)

(72) Inventors: Xueli Zhang, Hefei (CN); Dongzhu Zhang, Hefei (CN)

(73) Assignee: ANHUI HUAHENG BIOENGINEERING CO., LTD,, Hefei (CN)

(21) Appl. No.: 14/395,187

(22) PCT Filed: Dec. 28, 2012
(86) PCT No.: PCT/CN2012/001760

§ 371 Date: Oct. 17, 2014

(51) Int. Cl. C12P 13/06; C12N 15/70; C12N 9/90; C12R 1/19; C12N 9/06

(52) U.S. Cl. CPC C12P 13/06; C12R 1/19; C12N 9/0016; C12N 9/90; C12N 15/70; C12Y 104/01001; C12Y 501/01001

(57) ABSTRACT

The present invention discloses a DL-alanine-producing engineering bacterium. This DL-alanine-producing engineering bacterium itself was inactivated in lactate dehydrogenase, pyruvate formate lyase, alcohol dehydrogenase, acetate kinase, fumarate reductase, alanine racemase, and methylglyoxal synthase; moreover, onto the chromosome thereof an exogenous L-alanine dehydrogenase gene and alanine racemase gene were integrated. In the present application, by integrating the exogenous L-alanine dehydrogenase gene into the chromosome of the engineering bacterium, the intermediate of glycolysis, pyruvic acid, was converted into L-alanine; by further integrating the exogenous alanine racemase gene, partial L-alanine was converted into D-alanine, achieving the direct production of DL-alanine from raw material saccharides, thereby decreasing the production cycle of DL-alanine, and increasing the yield of DL-alanine.

### FIELD OF THE INVENTION

The present invention relates to the production field of DL-alanine, particularly relates to a DL-alanine-producing engineering bacterium and methods of producing DL-alanine with the engineering bacterium.

### DESCRIPTION OF BACKGROUND

DL-alanine is mainly used in food processing industry, as a nutritional supplement and a flavoring, since it has good taste for enhancing the seasoning effects of a flavoring. It is also used in medicine industry for synthesizing some pesticides, medicines and medicine intermediates.

At present, DL-alanine is mainly produced with enzymatic catalysis technique (alanine racemase) or chemical racemization method; however, the chemical racemization method is gradually weeded out, because it needs organic acids as solvents, resulting in the defects such as environmental pollution and low yield. In the enzymatic catalysis technique, firstly, a glucose is fermented to produce L-alanine, which is then converted, through enzymatic catalysis, into D-alanine having an accumulation of 30˜50 g/L during this procedure, the yield is lower but the production cycle is long; secondly, strains should be subjected to high-density cultivation so as to secrete alanine racemases required for producing DL-alanine, the demand for oxygen is high during this process; in addition, since the alanine racemase gene is cloned into a plasmid for high-level expression, during cultivation of strains, antibiotics should be added in order to maintain stable genetic replication of the plasmid. Therefore, this solution is not easy to be industrialized either.

### DISCLOSURE OF THE INVENTION

The primary purpose of the present invention is to provide a DL-alanine-producing engineering bacterium, which engineering bacterium can directly produce DL-alanine using glucose, with high yield and short production cycle.

In order to achieve the above purpose, the present invention adopts the following technical solution: inactivating the lactate dehydrogenase gene, pyruvate formate lyase gene, alcohol dehydrogenase gene, acetate kinase gene, fumarate reductase gene, alanine racemase gene, and methylglyoxal synthase gene on the chromosome of an original bacterium, into whose chromosome, an exogenous L-alanine dehydrogenase gene and an exogenous alanine racemase gene are integrated, and performing a screening to obtain the DL-alanine-producing engineering bacterium. Said inactivation method includes knockout, insertion mutation, or interfering the expression of said genes with small RNAs.

Since a bacterium experiences L-alanine anabolism in metabolism, the solution as defined above can be achieved in all conventional bacteria. The present application preferably selects Escherichia coli as the construction bacterium of the DL-alanine-producing engineering bacterium.

Said exogenous L-alanine dehydrogenase gene was derived from Geobacillus stearothermophilus; said exogenous alanine racemase gene was derived from Bacillus subtilis, preferably from Bacillus subtilis 168; and inactivation of the alanine racemase mainly refers to the alanine racemase (DadX) gene of the E. coli itself getting knocked out.

In the above mentioned construction solution, the alaR gene was directly integrated into the Escherichia coli XZ-A26 strain (CN 102329765A), which produces L-alanine. The XZ-A26 strain was deposited, on Jul. 26, 2010, with the accession number of CGMCC No. 4036, in China General Microbiological Culture Collection Center of the Institute of Microbiology, Chinese Academy of Sciences, located at No. 3, Court 1, Beichen West Road, Chaoyang District, Beijing. The XZ-A26 strain was inactivated in lactate dehydrogenase, pyruvate formate lyase, alcohol dehydrogenase, acetate kinase, fumarate reductase, and alanine racemase, and onto the chromosome thereof, an exogenous L-alanine dehydrogenase gene had been integrated. Thus, it only needs to integrate an exogenous alanine racemase gene into the chromosome of the XZ-A26 strain, and knock out the methylglyoxal synthase gene. If the original bacterium is used to construct the DL-alanine-producing engineering bacterium of the present invention, the lactate dehydrogenase gene, pyruvate formate lyase gene, alcohol dehydrogenase gene, acetate kinase gene, fumarate reductase gene, alanine racemase gene, and methylglyoxal synthase gene on the chromosome of the bacterium itself may be knocked out, and the exogenous L-alanine dehydrogenase and alanine racemase genes may be integrated, so as to achieve the same effects. The method for knocking out and integrating the above genes may be carried out in accordance with the technical solution as recorded in the Chinese patent titled “an XZ-A26 strain for producing L-alanine with high yield and construction method as well as uses of the same” (CN 102329765 A).

The construction method of the DL-alanine-producing engineering bacteria of the present invention particularly comprises integrating the exogenous alanine racemase gene into the chromosome of Escherichia coli XZ-A26 CGMCC No. 4036, serving as an original strain, at the methylglyoxal synthase gene locus. Said exogenous alanine racemase gene has a sequence as set forth by SEQ ID NO. 14 in the sequence listing; said methylglyoxal synthase gene has a sequence of nucleotides at positions 495-953 starting from the 5′ end of SEQ ID NO. 15 in the sequence listing; and the artificial regulatory element M1-93 has a sequence as set forth by SEQ ID NO. 17 in the sequence listing.

The DL-alanine-producing engineering bacteria of the present invention were constructed following the steps as below:

(a) cloning and integration of the alanine racemase gene:
1) constructing a DNA fragment I, consisted of a upstream arm of the methylglyoxal synthase gene mgsA, a chloramphenicol gene, a levansucrase gene and a downstream arm of the methylglyoxal synthase gene mgsA linked in tandem in the above order, which DNA fragment I was subjected to electric shock and transformed into Escherichia coli XZ-A26 CGMCC No. 4036 bearing a pKD46 plasmid; and screening chloramphenicol-resistant colonies, which were identified with a pair of primers, consisted of a DNA fragment having a sequence of SEQ ID NO: 3 and a DNA fragment having a sequence of SEQ ID NO: 4, to give a strain with an amplification product of 4111 bp, designated as XZ-A27;
2) constructing a DNA fragment II, consisted of a upstream arm of the methylglyoxal synthase gene mgsA, a lac′ gene, a trc promoter, the alanine racemase gene alaR of Bacillus subtilis 168 and a downstream arm of the methylglyoxal synthase gene mgsA linked in tandem in the above order, which DNA fragment II was subjected to electric shock, transformed into XZ-A27 bearing the pKD46 plasmid, and cultivated with LB medium containing sucrose but free of sodium chloride; and screening strain which has amplification product of 4210 bp identified with the primer pair consisted of a DNA fragment having a sequence of SEQ ID NO: 3 and a DNA fragment having a sequence of SEQ ID NO: 4; and designating the strain as XZ-A28;
(b) regulation of the alanine racemase gene alaR:
3) constructing a DNA fragment III, which was subjected to electric shock, transformed into XZ-A28 bearing the pKD46 plasmid, and identified with a pair of primers, consisted of a DNA fragment having a sequence of SEQ ID NO: 11 and a DNA fragment having a sequence of SEQ ID NO: 13, to give a strain with an amplification product being 1890 bp, i.e., the DL-alanine-producing engineering bacterium;
wherein, particularly, the DNA fragment I was obtained with the following method: the genomic DNA of E. coli ATCC 8739, serving as a template, was amplified with the primer pair as set forth by SEQ ID NO: 3 and SEQ ID NO: 4, to give the methylglyoxal synthase gene mgsA and the upstream and downstream fragments thereof, and then, the amplification products were cloned into a pEASY-Blunt cloning Vector, giving a kanamycin-resistant plasmid, pXZ-A19; with the DNA fragments as set forth by SEQ ID NO: 5 and SEQ ID NO: 6 as primers, the pXZ-A19 was amplified to give a product, which was linked with a DNA fragment containing the chloramphenicol gene and levansucrase gene, giving a plasmid, pXZ-A20, then, plasmid DNA of the pXZ-A20 was used as a template to amplify, with the DNA fragments as set forth by SEQ ID NO: 3 and SEQ ID NO: 4 as primers, to give the DNA fragment I; wherein, the DNA fragment containing the chloramphenicol gene and levansucrase gene was a DNA fragment achieved by an amplification conducted on a pLOI4162 plasmid with the primer pair as set forth by SEQ ID NO: 7 and SEQ ID NO: 8;
the DNA fragment II was obtained with the following method: the genomic DNA of Bacillus subtilis 168 was amplified with the primers as set forth by SEQ ID NO: 1 and SEQ ID NO: 2 to give fragments of the alanine racemase gene, which were then inserted between the XbaI and SalI restriction sites of a pTrc99A plasmid, giving a plasmid, pTrc99A-alaR; a product amplified from pXZ-A19 with the DNA fragments as set forth by SEQ ID NO: 5 and SEQ ID NO: 6 as primers and a product amplified from pTrc99A-alaR with the primers as set forth by SEQ ID NO: 9 and SEQ ID NO: 10 were linked to give a plasmid, pXZ-A21, afterwards, plasmid DNA of the pXZ-A21 was amplified with the DNA fragments as set forth by SEQ ID NO: 3 and SEQ ID NO: 4 as primers, resulting in the DNA fragment II;
the DNA fragment III was obtained with the following method: an amplification was conducted on the genomic DNA of the recombinant E. coli strain M1-93, with the sequences as shown by the DNA fragments of SEQ ID NO: 11 and SEQ ID NO: 12 as primers, resulting in the DNA fragment III (the sequences were as shown by SEQ ID NO: 16 in the sequence listing).

In a more particular solution, the DL-alanine-producing engineering bacterium was a Escherichia coli XZ-A30 strain, deposited, on Oct. 12, 2012, with the accession number of CGMCC No. 6667, in China General Microbiological Culture Collection Center (CGMCC) of the Institute of Microbiology, Chinese Academy of Sciences, located at NO. 3, Court 1, Beichen West Road, Chaoyang District, Beijing. This bacterium was able to be fermented to produce high concentrations of DL-alanine; moreover, the ratio of optical purity between D-alanine and L-alanine was 50:50.

As shown in FIG. 1, in the present application, by integrating the exogenous L-alanine dehydrogenase gene into the chromosome of the engineering bacterium, the intermediate of glycolysis, pyruvic acid, was converted into L-alanine; by further integrating the exogenous alanine racemase gene, partial L-alanine was converted into D-alanine, achieving the direct production of DL-alanine from raw material saccharides, thereby decreasing the production cycle of DL-alanine. Meanwhile, the engineering bacterium itself was inactivated in each of the lactate dehydrogenase, methylglyoxal synthase, pyruvate formate lyase, alcohol dehydrogenase, acetate kinase, and fumarate reductase, avoiding synthesizing of byproducts, such as lactic acid, formic acid, ethanol, acetic acid, and succinic acid, during metabolism, such that the raw material saccharides metabolized according to defined pathways to synthesize DL-alanine, thereby increasing the yield of DL-alanine. Herein, the DL-alanine refers to D-alanine and L-alanine. Introduction of the exogenous alanine racemase gene mainly served the purpose for enabling half of the L-alanine as produced to be converted into D-alanine, such that the optical purity ratio between L-alanine and D-alanine in the products resulting from fermentation was 50:50, thereby meeting the production needs of the products.

Another purpose of the present invention is to provide a method for producing DL-alanine with the engineering bacterium, which adopts the following solution:

a method for producing DL-alanine, comprising fermenting and cultivating the strains of the above constructed DL-alanine-producing engineering bacteria, under anaerobic/aerobic cultivation conditions, at a cultivation temperature of 30˜42° C., and a pH controlled between 6.5˜7.5, separating and extracting DL-alanine.

The medium for fermenting and cultivating was consisted of raw material saccharides, nitrogen sources and trace inorganic salts, wherein, the raw material saccharides were selected from one or any combination of two or more of glucose, sucrose, fructose, xylose, maltose, lactose, galactose, Manihot esculenta, Zea mays, Beta vulgaris, lignocellulose or the hydrolysates and syrups thereof; the nitrogen sources were inorganic nitrogen-containing compounds, selected from one or any combination of two or more of ammonium chloride, ammonium acetate, ammonium sulfate and ammonium phosphate; the trace inorganic salts were selected from one or any combination of two or more of soluble iron salts, cobalt salts, copper salts, zinc salts, manganese salts and molybdate; said medium was preferably consisted of 120 g/L of glucose, 4 g/L of ammonium chloride, 5 g/L of NaH₂PO₄, 5 g/L of Na₂HPO₄, 1 g/L of MgSO₄.7H₂O, 0.1 g/L of CaCl₂ 2H₂O and 4 ml/L of trace inorganic salts, wherein, the trace inorganic salts comprised 1.5 mg of FeCl₃.6H₂O, 0.1 mg of CoCl₂.6H₂O, 0.1 mg of CuCl₂.2H₂O, 0.1 mg of ZnCl₂, 0.1 mg of Na₂MoO₄.2H₂O, and 0.2 mg of MnCl₂.4H₂O₂, which were diluted with distilled water to volume of 1 L, and filtered for sterilization.

The duration for said fermenting and cultivating was 40-60 hours; said engineering bacteria were subjected to seed cultivation prior to said fermenting and cultivating, at a temperature of 30° C., a rotational speed of the shaker of 50 r/min (50 rotations/min), for 18 h.

### BRIEF DESCRIPTION OF THE DRAWINGS

FIG. 1 shows the metabolic pathway of the DL-alanine-producing engineering bacteria.

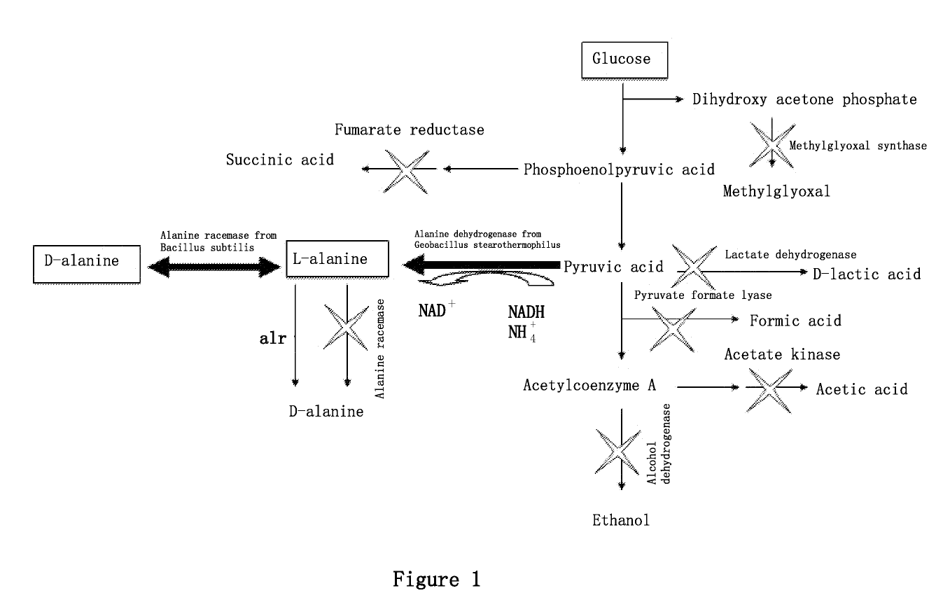

FIG. 2 shows the schematic diagram of plasmid pXZ-A19.

FIG. 3 shows the schematic diagram of plasmid pXZ-A20.

FIG. 4 shows the schematic diagram of plasmid pXZ-A21.

FIG. 5 shows the component spectrum of the fermentation broth of XZ-A30 strain determined with high performance liquid chromatograph.

### BEST MODE FOR CARRYING OUT THE INVENTION

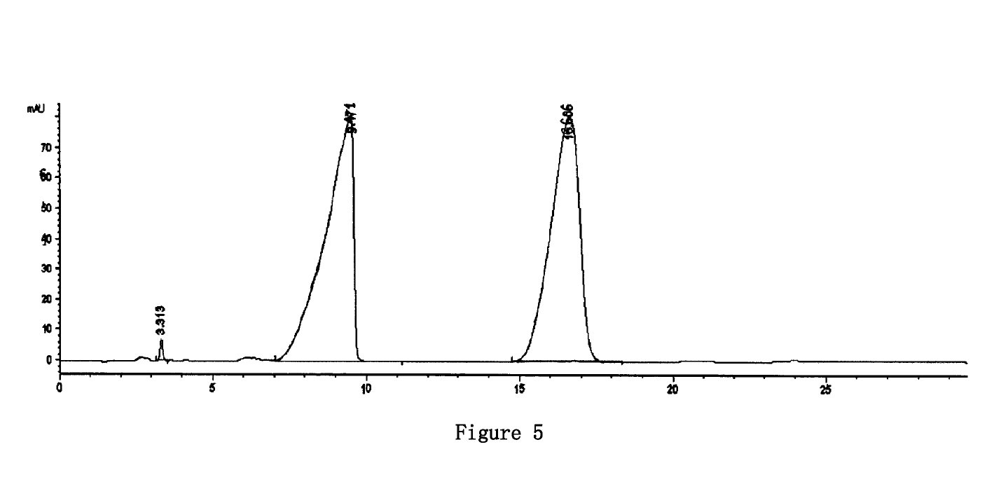

The following examples are presented for further describing the present invention, which, however, are not intended to limit the substantive contents of the present invention. Each of the experimental methods used in the following examples is a conventional method, unless otherwise indicated. All of the materials, reagents and the like used in the following examples are commercially available, unless otherwise indicated. Example 1 is an example for constructing the DL-alanine-producing engineering bacterium, and Examples 2˜5 are examples for producing DL-alanine using the engineering bacterium as constructed in Example 1. In Examples 2˜5, components of the fermentation broth were determined with high performance liquid chromatograph, Agilent-1200; the quantification and chirality determination of DL-alanine were conducted with ligand-exchange chiral isomer liquid chromatographic column (Chiralpak MA(+)) from Daciel; and the remaining glucoses and miscellaneous acids in the fermentation broth were determined with Aminex HPX-87H carbohydrate analysis column from Biorad. The contents of the components of the fermentation broth resulting from Examples 2˜5 as determined by the high performance liquid chromatograph were shown in FIG. 5. In the following Examples, quantification was performed using external standard method (standard curve method), with D-alanine and L-alanine as standards.

### Example 1

### Construction of the XZ-A30 Strain

The construction of XZ-A30 strain comprised two steps, (a) and (b), in particular:

### (a) Cloning and Integration of the Alanine Racemase Gene

The cloning and integration of the alanine racemase gene alaR were conducted in the following two steps:

(a1) Cloning of the alaR Gene

The genomic DNA of Bacillus subtilis 168 (Moszer I, Jones L M, Moreira S, Fabry C, Danchin A. SubtiList: the reference database for the Bacillus subtilis genome. Nucleic Acids Res. 2002, 30(1):62-65, available from Anhui Huaheng Bioengineering Co., Ltd) was used as a template to amplify, with the primers alaR up-XbaI/alaR down-SalI, the alanine racemase gene alaR of Bacillus subtilis (SEQ ID NO: 14). The primer sequences were:

alaR up-XbaI:
(SEQ ID NO: 1)
GGAGAGTCTAGAATGAGCACAAAACCTTT;
alaR down-SalI:
(SEQ ID NO: 2)
CGCTGCGTCGACTTAATTGCTTATATTTACC.

The amplification system comprised a total of 50 μl volume containing: Stratagene PfuUltra 10× buffer, 5 μl; dNTP (10 mM each dNTP), 1 μl; DNA template, 20 ng; primers (10 μl), 1 μl; PfuUltra (2.5 U/μl), 1 μl; and distilled water, 40 μl. The amplification protocol consisted of 2 min initial denaturation at 95° C. (1 cycle); followed by denaturation at 95° C. for 30 s, annealing at 55° C. for 30 s, extension at 72° C. for 2 min (30 cycles); and a final extension at 72° C. for 10 min (1 cycle). The amplification products were cloned into the pTrc99A plasmid (Amann, E., Ochs, B. and Abel, K. J. Tightly regulated tac promoter vectors useful for the expression of unfused and fused proteins in Escherichia coli. Gene. 1988, 69:301-15, available from Anhui Huaheng Bioengineering Co., Ltd) at the XbaI and SalI restriction sites, generating plasmid, pTrc99A-alaR.

Wherein, cloning was carried out as below: the PCR fragments of alaR gene resulting from amplification with primers alaR up-XbaI/alaR down-SalI were washed and purified, generating a DNA fragment containing the alaR gene (1192 bp, containing SEQ ID NO: 14 and sequences introduced by primers 1 and 2). The enzymatic digestion system included: 0.2 μg of alaR DNA fragment, 2 μl of 10* FastDigest Green buffer (Thermo Scientific Company), 1 μl of FastDigest XbaI (Thermo Scientific Company), and 1 μl of FastDigest SalI (Thermo Scientific Company), which were supplemented with distilled water to make a final volume of 20 μl; the system was incubated at 37° C. for 10 min. After that, digested DNA fragments containing alaR (SEQ ID NO: 14) were recovered from agarose gels. The enzymatic digestion system of plasmid pTrc99A included: 1 μg of plasmid pTrc99A, 2 μl of 10* FastDigest Green buffer (Thermo Scientific Company), 1 μl of FastDigest XbaI (Thermo Scientific Company), and 1 μl of FastDigest SalI (Thermo Scientific Company), supplemented with distilled water to make a final volume of 201; the system was incubated at 37° C. for 10 min. Then, digested DNA fragments containing pTrc99A were recovered from agarose gels. The ligation system included: 10 ng of pTrc99A fragments recovered from enzymatic digestion, 20 ng of alaR DNA fragments, and 2 μl of Quick Ligation Reaction Buffer, which was supplemented with distilled water to make a final volume of 10 μl, followed by 0.5 μl of Quick T4 DNA Ligase, and placed at 25° C. for 10 min, subsequently, 5 μl of which was added into 50 μl of Trans1-T1 competent cells (purchased from BeiJing TransGen Biotech Co., Ltd.). The mixture was ice bathed for 30 min, and placed on the ice for 2 min immediately after heat shock at 42° C. for 30 s, to which, and then, 2501 of LB medium was added prior to incubation at 200 rpm, 37° C. for 1 hour. 2001 bacteria solution was spread onto a LB plate containing ampicillin (final concentration, 100 μg/ml), cultivated overnight, and then, 5 positive mono-colonies were selected for liquid cultivation, from which plasmids of positive clones (plasmids cloning the alaR DNA fragments into pTrc99A) were isolated for sequencing. The sequencing results showed the digested pTrc99A plasmids were ligated with DNA fragments containing alaR, demonstrating that the plasmids were correctly constructed, which plasmids were designated as pTrc99A-alaR.

(a2) Integrating the alaR Gene into the Chromosome of the L-Alanine-Producing Bacterium XZ-A26 at the Methylglyoxal Synthase Gene mgsA

Integration of the alaR gene into the chromosome of the L-alanine-producing bacterium XZ-A26 CGMCC No. 4036 (Patent Publication No. CN102329765A, Anhui Huaheng Bioengineering Co., Ltd) at the methylglyoxal synthase gene mgsA was performed in the following six steps:

In the first step, the genomic DNA of E. coli ATCC 8739 (Zhang X, Jantama K, Shanmugam K T, Ingram L O. Re-Engineering Escherichia coli for succinate production in mineral salts medium. Appl Environ Microbiol. 2009, 75(24):7807-7813, available from Anhui Huaheng Bioengineering Co., Ltd) was amplified for the methylglyoxal synthase gene mgsA (GeneID:6064585) as well as about 400 bp bases upstream and downstream thereof, using primers mgsA-up/mgsA-down. The primer sequences were:

mgsA-up:
(SEQ ID NO: 3)
CAGCTCATCA ACCAGGTCAA;
mgsA-down:
(SEQ ID NO: 4)
AAAAGCCGTC ACGTTATTGG.

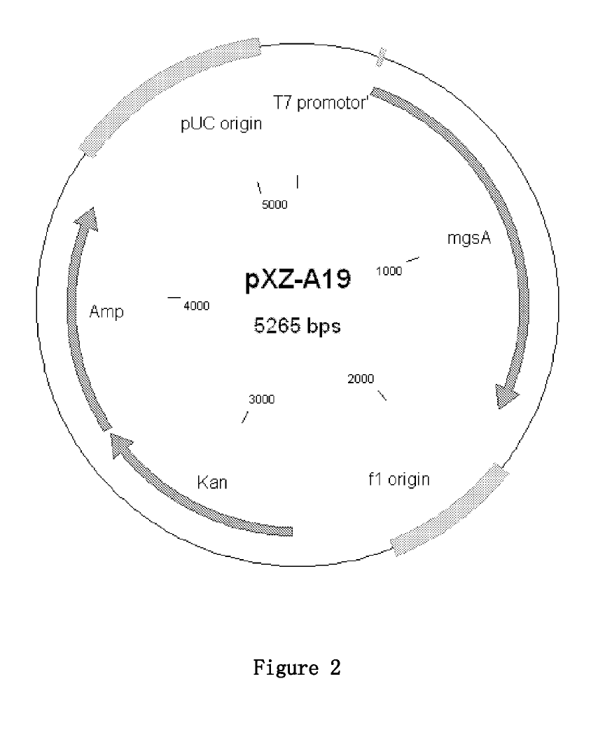

The amplification system included a total of 50 μl volume containing: NewEngland Biolabs Phusion 5× buffer, 10 μl; dNTPs (each dNTP was 10 mM) each 1 μl; DNA template, 20 ng; primers (10 μl), 41; Phusion High-Fidelity DNA polymerase (2.5 U/μl), 0.5 μl; and distilled water, 33.5 μl. The amplification protocol consisted of 2 min initial denaturation at 98° C. (1 cycle); followed by denaturation at 98° C. for 10 s, annealing at 59° C. for 10 s, extension at 72° C. for 90 s (30 cycles); and a final extension at 72° C. for 5 min (1 cycle). The PCR amplification generated DNA fragments, as set forth by SEQ ID NO. 15, containing the methylglyoxal synthase gene (nucleotides at positions 495-953 starting from the 5′ end of SEQ ID NO. 15 in the sequence listing), fragments of about 400 bp upstream of this gene (nucleotides at positions 1-494 starting from the 5′ end of SEQ ID NO. 15 in the sequence listing) and fragments of about 400 bp downstream of this gene (nucleotides at positions 954-1435 starting from the 5′ end of SEQ ID NO. 15 in the sequence listing). The amplification products were cloned into a pEASY-Blunt cloning Vector (purchased from BeiJing TransGen Biotech Co., Ltd.). The cloning system included: 1 μl of PCR amplification products, and 1 μl of pEASY-Blunt cloning Vector, which were gently mixed, reacted at room temperature for 5 min, and added into 50 μl of Trans1-T1 competent cells (purchased from BeiJing TransGen Biotech Co., Ltd.). The mixture was ice bathed for 30 min, and placed on the ice for 2 min immediately after heat shock at 42° C. for 30 s, to which, and then, 250 μl of LB medium was added prior to incubation at 200 rpm, 37° C. for 1 hour. 200 μl bacteria solution was spread onto a LB plate containing kanamycin (final concentration, 15 ug/ml), cultivated overnight, and then, 5 positive mono-colonies were selected for liquid cultivation, from which plasmids of positive clones were isolated for sequencing. The sequencing results showed the methylglyoxal synthase gene and fragments of about 400 bp bases upstream and downstream thereof were inserted into the vector pEASY-Blunt, demonstrating that the plasmid was correctly constructed; the resulting recombinant plasmid was designated as pXZ-A19 (FIG. 2).

In the second step, a DNA fragment was amplified from pXZ-A19 plasmid DNA, using primers mgsA-1/mgsA-3; the primer sequences were:

mgsA-1:
(SEQ ID NO: 5)
AGCGTTATCT CGCGGACCGT;
mgsA-3:
(SEQ ID NO: 6)
GCATTTGTTTGCAGTGATCG.

The amplification system included a total of 50 μl volume containing: NewEngland Biolabs Phusion 5× buffer, 10 μl; dNTPs (each dNTP was 10 mM), each 1 μl; DNA template, 20 ng; primers (10 μl), 41; Phusion High-Fidelity DNA Polymerase (2.5 U/μl), 0.5 μl; and distilled water, 33.5 μl. The amplification protocol consisted of 2 min initial denaturation at 98° C. (1 cycle); followed by denaturation at 98° C. for 10 s, annealing at 60° C. for 10 s, extension at 72° C. for 2 min (30 cycles); and a final extension at 72° C. for 5 min (1 cycle). The PCR amplification products comprised the pEASY-Blunt vector and about 400 bp bases upstream and downstream of the methylglyoxal synthase gene, that is, the pEASY-Blunt vector and a nucleotide sequence at positions 925-1435 starting from the 5′ end of SEQ ID NO. 15 as well as a nucleotide sequence at positions 1-570 starting from the 5′ end of SEQ ID NO. 15.

In the third step, a DNA fragment containing the chloramphenicol gene (cat) and levansucrase gene (sacB) was ligated to the PCR amplification product of the second step. PCR amplification was conducted on a pLO14162 plasmid (Jantama K, Zhang X, Moore J C, Shanmugam K T, Svoronos S A, Ingram L O. Eliminating side products and increasing succinate yields in engineered strains of Escherichia coli C. Biotechnol Bioeng. 2008, 101(5): 881-893., available from Anhui Huaheng Bioengineering Co., Ltd) to amplify cat-sacB fragments using primers cat-sacB-up/down cat-sacB. The primer sequences were:

cat-sacB-up:
(SEQ ID NO: 7)
GGAGAAAATACCGCATCAGG;
cat-sacB-down:
(SEQ ID NO: 8)
GCGTTGGCCGATTCATTA.

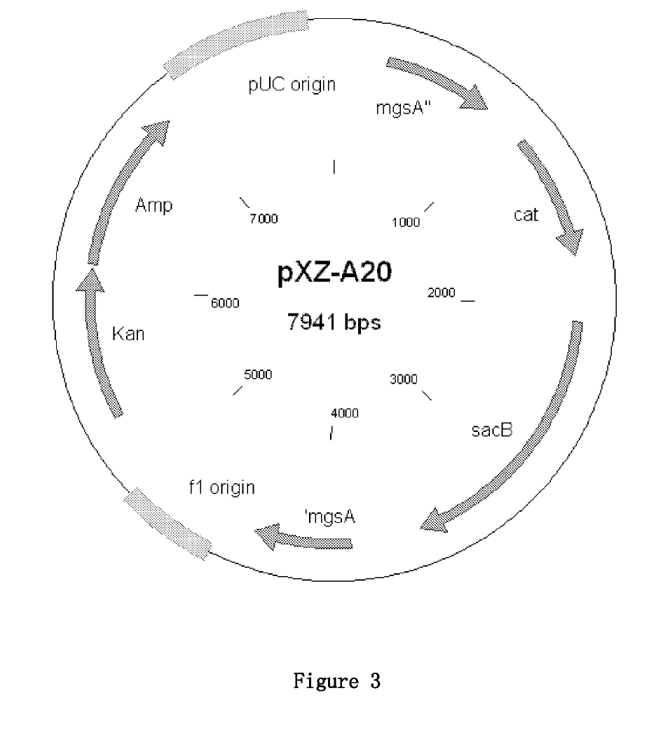

The amplification system was same as that in the first step. A DNA fragment (3030 bp) containing the chloramphenicol gene (cat) and levansucrase gene (sacB) was recovered from agarose gel electrophoresis. The DNA fragment containing the chloramphenicol gene (cat) and levansucrase gene (sacB) was ligated to the PCR amplification product of the second step, using the following ligation system: 10 ng of the PCR amplification product of the second step, 30 ng of cat-sacB DNA fragment, 2 μl of 10×T4 ligation buffer (NEB Company), 1 μl of T4 ligase (NEB Company, 400,000 cohesive end units/ml), which were supplemented with distilled water to make a final volume of 20 μl, and ligated at room temperature for 2 hours. 5 μl of the mixture was taken to be added into 50 μl of Trans1-T1 competent cells (purchased from BeiJing TransGen Biotech Co., Ltd.), ice bathed for 30 min, and placed on the ice for 2 min immediately after heat shock at 42° C. for 30 s, to which, and then, 250 μl of LB medium was added prior to incubation at 200 rpm, 37° C. for 1 hour. 200 μl bacteria solution was spread onto a LB plate containing chloramphenicol (final concentration, 17 ug/ml), cultivated overnight, and then, 5 positive mono-colonies were selected for liquid cultivation, from which plasmids of positive clones (plasmids cloning the cat-sacB DNA fragments into pXZ-A19) were isolated for sequencing. The sequencing results showed the cat-sacB DNA fragments were ligated to the PCR amplification products of the above second step, demonstrating that the plasmids were correctly constructed; the resulting recombinant plasmids were designated as pXZ-A20 (FIG. 3).

In the fourth step, plasmid DNA of pXZ-A20 was amplified with primers mgsA-up/mgsA-down (SEQ ID NO: 3/SEQ ID NO: 4) to generate DNA fragment I, using the following amplification system in a total of 50 μl volume containing: NewEngland Biolabs Phusion 5× buffer, 10 μl; dNTPs (each dNTP was 10 mM), each 1 μl; DNA template, 20 ng; primers (10 μM), 2 μl; Phusion High-Fidelity DNA polymerase (2.5 U/μl), 1 μl; and distilled water, 33.5 μl. The amplification protocol consisted of 2 min initial denaturation at 98° C. (1 cycle); followed by denaturation at 98° C. for 10 s, annealing at 59° C. for 10 s, extension at 72° C. for 100 s (30 cycles); and a final extension at 72° C. for 5 min (1 cycle). The DNA fragment I, resulting from the amplification, was consisted of about 400 base pairs upstream of the methylglyoxal synthase gene mgsA (the sequence of nucleotides at positions 1-570 starting from the 5′ end of SEQ ID NO. 15), a cat-sacB DNA fragment, about 400 base pairs downstream of the methylglyoxal synthase gene mgsA (the sequence of nucleotides at positions 925-1435 starting from the 5′ end of SEQ ID NO. 15), which were linked in the above order.

The DNA fragment I was subjected to the first homologous recombination. Firstly, the pKD46 plasmid (Dower et al., 1988; Dower, W. J., Miller, J. F., Ragsdale, C. W.1988. High efficiency transformation of E. coli by high voltage electroporation. Nucleic Acids Res. 16: 6127-6145, available from Anhui Huaheng Bioengineering Co., Ltd) was transformed into E. coli ATCC8739 bp calcium chloride transformation. And then, the DNA fragment I was subjected to electric shock and transformed into the E. coli ATCC8739 carrying pKD46.

The electric shock transformation comprised the following conditions: firstly, electransformed competent cells of E. coli ATCC8739 bearing pKD46 plasmids were prepared; 50 μl of the competent cells was placed on ice, into which, 50 ng of DNA fragment I was added; then, the mixture was placed on ice for 2 min, and transferred into a 0.2 cm Bio-Rad cuvette. The MicroPulser (Bio-Rad Company) electroporation apparatus was used, with the voltage, an electric shock parameter, of 2.5 kv. 1 ml of the LB medium was transferred into the cuvette immediately after the electric shock, blowed and flicked 5 times, transferred into a test tube, and incubated at 75 rpm, 30° C. for 2 hours. 200 μl of the bacteria solution was spread onto a LB plate containing chloramphenicol (final concentration, 17 μg/ml), cultivated at 37° C. overnight, and then, 5 mono-colonies were selected for verification by PCR amplification (using primers mgsA-up/mgsA-down (SEQ ID NO: 3/SEQ ID NO: 4), and the correct colony amplification product was of 4111 bp. One correct mono-colony was selected, and designated as XZ-A27, wherein, the methylglyoxal synthase gene on the chromosome was substituted with the cat-sacB DNA fragment through homologous recombination.

In the fifth step, the plasmid pTrc99A-alaR was amplified with primers alaR-up/a/aR-down to generate the lacI gene and trc promoter as well as alanine racemase gene alaR on the pTrc99A plasmid vector, which were ligated to the PCR amplification products of the second step. The primer sequences were:

alaR-up:
(SEQ ID NO: 9)
GGCATGCATTTACGTTGACA;
alaR-down:
(SEQ ID NO: 10)
AGAAACGCAAAAAGGCCATC.

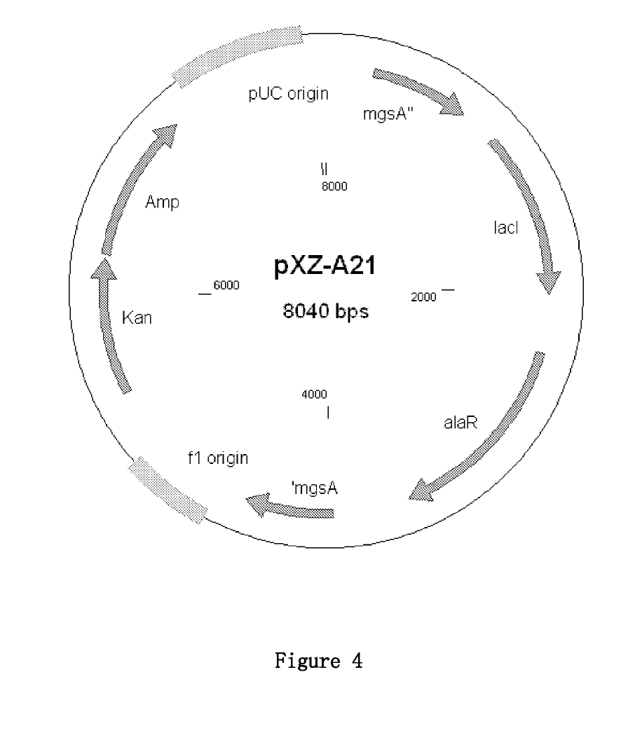

The cloning system was same as that of the third step. 200 μl of the bacteria solution was spread onto a LB plate containing kanamycin (final concentration, 15 μg/ml), cultivated overnight, and then, 5 positive mono-colonies were selected for liquid cultivation, from which plasmids of positive clones were isolated for sequencing. The sequencing results showed the alanine racemase gene alaR was inserted into the vector pXZ-A19, demonstrating that the plasmids were correctly constructed; the resulting plasmid was designated as pXZ-A21 (FIG. 4).

In the sixth step, the pXZ-A21 plasmid DNA was amplified with primers mgsA-up/mgsA-down (SEQ ID NO: 3/SEQ ID NO: 4) to generate DNA fragment II. The DNA fragment II was consisted of about 400 bases upstream of the methylglyoxal synthase gene mgsA (the sequence of nucleotides at positions 1-570 starting from the 5′ end of SEQ ID NO. 15), lacI gene, trc promoter, alanine racemase gene alaR, and about 400 bases downstream of the methylglyoxal synthase gene mgsA (the sequence of nucleotides at positions 925-1435 starting from the 5′ end of SEQ ID NO. 15), which were linked in the above order. DNA fragment II was subjected to the second homologous recombination. Firstly, the pKD46 plasmid was transformed into XZ-A27 by calcium chloride transformation, and then, DNA fragment II was subjected to electric shock and transformed into XZ-A27 bearing the pKD46 plasmid.

The electric shock transformation comprised the following conditions: firstly, electransformed competent cells of XZ-A27 bearing pKD46 plasmids were prepared; 50 μl of the competent cells was placed on ice, into which, 50 ng of DNA fragment II was added; then, the mixture was placed on ice for 2 min, and transferred into a 0.2 cm Bio-Rad cuvette. The MicroPulser (Bio-Rad Company) electroporation apparatus was used, with the voltage, an electric shock parameter, of 2.5 kv. 1 ml of the LB medium was transferred into the cuvette immediately after the electric shock, blowed and flicked 5 times, transferred into a test tube, and incubated at 75 rpm, 30° C. for 4 hours. The bacteria solution was transferred into a LB liquid medium present of 10% (mass percentage) sucrose but absent of sodium chloride (a 250 ml flask containing 50 ml medium), cultivated for 24 hours, and then, subjected to streak cultivation on a LB solid medium present of 6% (mass percentage) sucrose but absent of sodium chloride. As verified by PCR amplification (using primers mgsA-up/mgsA-down (SEQ ID NO: 3/SEQ ID NO: 4), the correct colony amplification product was a fragment of 4210 bp. One correct mono-colony was selected, and designated as XZ-A28, wherein, the cat-sacB DNA fragment on the chromosome was substituted with the lac′ gene, trc promoter and alanine racemase gene alaR through homologous recombination.

(b) Regulation of the Alanine Racemase Gene alaR

Regulation of the alanine racemase gene alaR was conducted in the following two steps:

In the first step, the genomic DNA of recombinant E. coli strain M1-93 (Lu J, Tang J L, Liu Y, Zhu X, Zhang T, Zhang X. Combinatorial modulation of galP and glk gene expression for improved alternative glucose utilization. Appl Microbiol Biotechnol. 2012, 93:2455-2462, available from Anhui Huaheng Bioengineering Co., Ltd) was amplified with primers mgsA-up-FRT/mgsA-alaR-FRT-down, to generate a DNA fragment III (having a sequence as shown by SEQ ID NO: 16 in the sequence listing) used in regulation of the expression of alaR gene; the primer sequences were:

mgsA-up-FRT:
(SEQ ID NO: 11)
ATGGAACTGACGACTCGCACTTTACCTGCGCGGAAACATATTGC
GCTGGTGTGTAGGCTGGAGCTGCTTC;
mgsA-alaR-FRT-down:
(SEQ ID NO: 12)
GACAAGTCAATTTCCGCCCACGTATCTCTGTAAAAAGGTTTTGT
GCTCATAGCTGTTTCCTGGTT.

In the second step: the DNA fragment III was elec-transformed into XZ-A28 carrying pKD46 plasmid.

The electransformation comprised the following conditions: firstly, electransformed competent cells of E. coli XZ-A28 bearing pKD46 plasmids were prepared; 50 μl of the competent cells was placed on ice, into which, 50 ng of DNA fragment III was added; then, the mixture was placed on ice for 2 min, and transferred into a 0.2 cm Bio-Rad cuvette. The MicroPulser (Bio-Rad Company) electroporation apparatus was used, with the voltage, an electric shock parameter, of 2.5 kv. 1 ml of the LB medium was transferred into the cuvette immediately after the electric shock, blowed and flicked 5 times, transferred into a test tube, and incubated at 75 rpm, 30° C. for 2 hours. 200 μl of the bacteria solution was spread onto a LB plate containing kanamycin (final concentration, 15 μg/ml), cultivated at 37° C. overnight, and then, 5 mono-colonies were selected for verification by PCR amplification (using primers mgsA-up-FRT/alaR-FRT-cexu (SEQ ID NO: 11/SEQ ID NO: 13); the primer sequences were:

alaR-FRT-cexu:
(SEQ ID NO: 13)
GCAGCGATTGCCACATACTC.

The correct colony amplification product was of 1890 bp. One correct mono-colony was selected, and designated as Escherichia coli XZ-A30 strain, wherein, the alanine racemase gene alaR was regulated by an artificial regulatory element M1-93 (SEQ ID NO: 17).

The Escherichia coli XZ-A30 strain was deposited, on Oct. 12, 2012, with the accession number of CGMCC No. 6667, in China General Microbiological Culture Collection Center (CGMCC) of the Institute of Microbiology, Chinese Academy of Sciences, located at No. 3, Court 1, Beichen West Road, Chaoyang District, Beijing.

This strain was able to be fermented to produce high concentrations of DL-alanine; moreover, the ratio of optical purity between D-alanine and L-alanine was 50:50.

The materials, reagents and the like used in construction of the above XZ-A30 strain each is commercially available, unless otherwise indicated. The plasmids as used (as shown in FIGS. 2, 3 and 4) and construction thereof are shown in Table 1.

TABLE 1
plasmids used in construction of the DL-alanine
engineering bacteria of the present invention

| Plasmids | Construction method |
|---|---|
| pTrc99A- | The alanine racemase gene was cloned into pTrc99A (alaR |
| alaR | up-XbaI/alaR down-SalI) |
| pXZ-A19 | The mgsA gene fragment of E. coli (mgsA-up/mgsA-down |
| | amplification) was cloned into pEASY-Blunt vector |
| pXZ-A20 | The Cat-sacB fragment was cloned into mgsA of pXZ-A19 |
| | (mgsA-1/mgsA-3) |
| pXZ-A21 | The alanine racemase gene was cloned into mgsA of |
| | pXZ-A19 (mgsA-1/mgsA-3) |

### Example 2

### Production of DL-Alanine by Anaerobic Fermentation Using XZ-A30 Strain

Seed medium and fermentation medium each comprised: glucose, 120 g/L; ammonium chloride, 4 g/L; NaH₂PO₄, 5 g/L; Na₂HPO₄, 5 g/L; MgSO₄.7H₂O, 1 g/L; CaCl₂.2H₂O, 0.1 g/L; and trace inorganic salts, 4 ml/L, with the pH of the medium being 6.5. The trace inorganic salts comprised 1.5 mg of FeCl₃.6H₂O, 0.1 mg of CoCl₂.6H₂O, 0.1 mg of CuCl₂.2H₂O, 0.1 mg of ZnCl₂, 0.1 mg of Na₂MoO₄.2H₂O, and 0.2 mg of MnCl₂.4H₂O₂, which were diluted with distilled water to volume 1 L, and filtered for sterilization.

150 ml of seed medium was loaded in a 250 ml conical flask, sterilized at 121° C. for 15 min, and cooled down, onto which, XZ-A30 was cultured at a cultivation temperature of 30° C. for 18 h, with the rotational speed of the shaker of 50 r/min (50 rotations/min), and then, used in inoculation of the fermentation medium.

A volume of 2.4 L fermentation medium was loaded in a 3 L fermentation tank, and sterilized at 121° C. for 15 min. The amount as inoculated was 0.1% (V/V), the fermentation temperature was 30° C., and the rotational speed of agitation was 100 rpm (100 rotations/min). The pH was controlled at 6.5 by ammonia liquor during fermentation, and the fermentation was lasted for 48 h.

Analysis method: components of the fermentation broth were determined with high performance liquid chromatograph, Agilent-1200; the quantification and chirality determination of DL-alanine were conducted with ligand-exchange chiral isomer liquid chromatographic column (Chiralpak MA(+)) from Daciel; and the remaining glucoses and miscellaneous acids in the fermentation broth were determined with Aminex HPX-87H carbohydrate analysis column from Biorad.

Results: contents of DL-alanine and organic acids in the fermentation broth: the concentration of DL-alanine was 114.6 g/L, wherein, D-alanine was 57.3 g/L, and L-alanine was 57.3 g/L, and the optical purity ratio between D-alanine and L-alanine was 50:50 (as shown in FIG. 5). The content of lactic acid was less than 0.1 g/L, the content of acetic acid was less than 0.1 g/L, the content of ethanol was less than 0.1 g/L, and the content of succinic acid was less than 0.1 g/L.

### Example 3

### Production of DL-Alanine by Anaerobic Fermentation Using XZ-A30 Strain

Seed medium and fermentation medium each comprised: glucose, 120 g/L; ammonium chloride, 4 g/L; NaH₂PO₄, 5 g/L; Na₂HPO₄, 5 g/L; MgSO₄.7H₂O, 1 g/L; CaCl₂.2H₂O, 0.1 g/L; and trace inorganic salts, 4 ml/L, with the pH of the medium being 6.5. The trace inorganic salts comprised 1.5 mg of FeCl₃.6H₂O, 0.1 mg of CoCl₂.6H₂O, 0.1 mg of CuCl₂.2H₂O, 0.1 mg of ZnCl₂, 0.1 mg of Na₂MoO₄.2H₂O, and 0.2 mg of MnCl₂.4H₂O₂, which were diluted with distilled water to volume 1 L, and filtered for sterilization.

150 ml of seed medium was loaded in a 250 ml conical flask, sterilized at 121° C. for 15 min, and cooled down, onto which, XZ-A30 was cultured at a cultivation temperature of 30° C. for 18 h, with the rotational speed of the shaker of 50 r/min (50 rotations/min), and then, used in inoculation of the fermentation medium.

A volume of 2.4 L fermentation medium was loaded in a 3 L fermentation tank, and sterilized at 121° C. for 15 min. The amount as inoculated was 0.1% (V/V), the fermentation temperature was 42° C., and the rotational speed of agitation was 100 rpm (100 rotations/min). The pH was controlled at 7.5 by ammonia liquor during fermentation, and the fermentation was lasted for 60 h.

Results: contents of DL-alanine and organic acids in the fermentation broth: the concentration of DL-alanine was 83.2 g/L, wherein, D-alanine was 41.6 g/L, and L-alanine was 41.6 g/L, and the optical purity ratio between D-alanine and L-alanine was 50:50. The content of lactic acid was less than 0.1 g/L, the content of acetic acid was less than 0.1 g/L, the content of ethanol was less than 0.1 g/L, and the content of succinic acid was less than 0.1 g/L.

### Example 4

### Production of DL-Alanine by Aerobic Fermentation Using XZ-A30 Strain

Seed medium and fermentation medium each comprised: glucose, 120 g/L; ammonium chloride, 4 g/L; NaH₂PO₄, 5 g/L; Na₂HPO₄, 5 g/L; MgSO₄.7H₂O, 1 g/L; CaCl₂.2H₂O, 0.1 g/L; and trace inorganic salts, 4 ml/L, with the pH of the medium being 6.5. The trace inorganic salts comprised 1.5 mg of FeCl₃.6H₂O, 0.1 mg of CoCl₂.6H₂O, 0.1 mg of CuCl₂.2H₂O, 0.1 mg of ZnCl₂, 0.1 mg of Na₂MoO₄.2H₂O, and 0.2 mg of MnCl₂.4H₂O₂, which were diluted with distilled water to volume 1 L, and filtered for sterilization.

150 ml of seed medium was loaded in a 250 ml conical flask, sterilized at 121° C. for 15 min, and cooled down, onto which, XZ-A30 was cultured at a cultivation temperature of 30° C. for 18 h, with the rotational speed of the shaker of 50 r/min (50 rotations/min), and then, used in inoculation of the fermentation medium.

A volume of 2.4 L fermentation medium was loaded in a 3 L fermentation tank, and sterilized at 121° C. for 15 min. The amount as inoculated was 0.1% (V/V), the fermentation temperature was 30° C., the rotational speed of agitation was 100 rpm (100 rotations/min), and the ventilation (air) was 0.1 L/min·L. The pH was controlled at 6.5 by ammonia liquor during fermentation, and the fermentation was lasted for 40 h.

Results: contents of DL-alanine and organic acids in the fermentation broth: the concentration of DL-alanine was 110.8 g/L, wherein, D-alanine was 55.4 g/L, and L-alanine was 55.4 g/L, and the optical purity ratio between D-alanine and L-alanine was 50:50. The content of lactic acid was less than 0.1 g/L, the content of acetic acid was less than 0.1 g/L, the content of ethanol was less than 0.1 g/L, and the content of succinic acid was less than 0.1 g/L.

### Example 5

### Production of DL-Alanine by Aerobic Fermentation Using XZ-A30 Strain

Seed medium and fermentation medium each comprised: glucose, 120 g/L; ammonium chloride, 4 g/L; NaH₂PO₄, 5 g/L; Na₂HPO₄, 5 g/L; MgSO₄.7H₂O, 1 g/L; CaCl₂.2H₂O, 0.1 g/L; and trace inorganic salts, 4 ml/L, with the pH of the medium being 6.5. The trace inorganic salts comprised 1.5 mg of FeCl₃.6H₂O, 0.1 mg of CoCl₂.6H₂O, 0.1 mg of CuCl₂.2H₂O, 0.1 mg of ZnCl₂, 0.1 mg of Na₂MoO₄.2H₂O, and 0.2 mg of MnCl₂.4H₂O₂, which were diluted with distilled water to volume 1 L, and filtered for sterilization.

150 ml of seed medium was loaded in a 250 ml conical flask, sterilized at 121° C. for 15 min, and cooled down, onto which, XZ-A30 was cultured at a cultivation temperature of 30° C. for 18 h, with the rotational speed of the shaker of 50 r/min (50 rotations/min), and then, used in inoculation of the fermentation medium.

A volume of 2.4 L fermentation medium was loaded in a 3 L fermentation tank, and sterilized at 121° C. for 15 min. The amount as inoculated was 0.1% (V/V), the fermentation temperature was 42° C., the rotational speed of agitation was 100 rpm (100 rotations/min), and the ventilation (air) was 0.1 L/min·L. The pH was controlled at 7.5 by ammonia liquor during fermentation, and the fermentation was lasted for 54 h.

Results: contents of DL-alanine and organic acids in the fermentation broth: the concentration of DL-alanine was 80.4 g/L, wherein, D-alanine was 40.2 g/L, and L-alanine was 40.2 g/L, and the optical purity ratio between D-alanine and L-alanine was 50:50. The content of lactic acid was less than 0.1 g/L, the content of acetic acid was less than 0.1 g/L, the content of ethanol was less than 0.1 g/L, and the content of succinic acid was less than 0.1 g/L.

### INDUSTRIAL APPLICATION

The process for producing DL-alanine with the engineering bacteria of the present invention is simple, which only needs to add, in the initial stage, into the fermentation tank raw material saccharides such as glucose and inorganic salts, and inoculate small amount of the engineering bacteria to ferment and produce DL-alanine. In the present application, fermentation was preferably conducted under anaerobic cultivation conditions, under which, fermenting DL-alanine may give a yield up to 114.6 g/L, increasing the yield of DL-alanine and reducing energy consumption; in addition, no antibiotic was added during fermentation, saving the cost of raw materials while improving the quality of products.

1. A DL-alanine-producing engineering bacterium, characterized in that: each of the lactate dehydrogenase gene, pyruvate formate lyase gene, alcohol dehydrogenase gene, acetate kinase gene, fumarate reductase gene, alanine racemase gene and methylglyoxal synthase gene on chromosome of an original bacterium is inactivated; and onto the chromosome thereof an exogenous L-alanine dehydrogenase gene and exogenous alanine racemase gene are integrated, and then, the DL-alanine-producing engineering bacterium is obtained by screening.

2. The engineering bacterium according to claim 1, characterized in that: said original bacterium is Escherichia coli; and the inactivation method comprises knockout, insertion mutation, or interfering the expression of said gene with small RNA.

3. The engineering bacterium of claim 1, characterized in that: said exogenous L-alanine dehydrogenase gene is derived from Geobacillus stearothermophilus; and said exogenous alanine racemase gene is derived from Bacillus subtilis.

4. The engineering bacterium according to claim 3, characterized in that: said exogenous alanine racemase gene is derived from Bacillus subtilis 168.

5. The engineering bacterium according to claim 4, characterized in that: construction method of the DL-alanine-producing engineering bacterium comprises integrating the exogenous alanine racemase gene into the chromosome of Escherichia coli XZ-A26 CGMCC No. 4036, serving as an original strain, at methylglyoxal synthase gene locus, and regulating expression of the exogenous alanine racemase gene with artificial regulatory element M1-93; wherein, said exogenous alanine racemase gene has a sequence as set forth by SEQ ID NO. 14 in sequence listing, said methylglyoxal synthase gene has a sequence of nucleotides at positions 495-953 starting from the 5′ end of SEQ ID NO. 15 in the sequence listing; and said artificial regulatory element M1-93 has a sequence as set forth by SEQ ID NO. 17 in the sequence listing.

6. The engineering bacterium according to claim 5, characterized in that: the DL-alanine-producing engineering bacterium is constructed following steps as below:
1) constructing a DNA fragment I, consisted of a upstream fragment of methylglyoxal synthase gene mgsA, a chloramphenicol gene, a levansucrase gene and a downstream fragment of the methylglyoxal synthase gene mgsA linked in tandem in the above order, said DNA fragment I is subjected to electric shock and transformed into Escherichia coli XZ-A26 CGMCC No. 4036 bearing a pKD46 plasmid; and screening chloramphenicol-resistant colonies, which are identified with a pair of primers, consisted of a DNA fragment having a sequence of SEQ ID NO: 3 and a DNA fragment having a sequence of SEQ ID NO: 4, to give a strain with an amplification product being 4111 bp, designated as XZ-A27; wherein the DNA fragment I is obtained with following method: the genomic gene of E. coli ATCC 8739, serving as a template, is amplified with the primer pair as set forth by SEQ ID NO: 3 and SEQ ID NO: 4, to give the methylglyoxal synthase gene mgsA and the upstream and downstream fragments thereof, and then, the amplification products are cloned into a pEASY-Blunt cloning vector, giving a kanamycin-resistant plasmid, pXZ-A19; with the DNA fragments as set forth by SEQ ID NO: 5 and SEQ ID NO: 6 as primers, the pXZ-A19 was amplified to give a product, which is linked with a DNA fragment containing the chloramphenicol gene and levansucrase gene, giving a plasmid, pXZ-A20, then, plasmid DNA of the pXZ-A20 is used as a template to amplify, with the DNA fragments as set forth by SEQ ID NO: 3 and SEQ ID NO: 4 as primers, the DNA fragment I; wherein, the DNA fragment containing the chloramphenicol gene and levansucrase gene is a DNA fragment obtained by amplifying pLOI4162 as templet with a primer pair as set forth by SEQ ID NO: 7 and SEQ ID NO: 8;
2) constructing a DNA fragment II, consisted of a upstream arm of the methylglyoxal synthase gene mgsA, a lad gene, a trc promoter, alanine racemase gene alaR of Bacillus subtilis 168 and a downstream arm of the methylglyoxal synthase gene mgsA linked in tandem in the above order, said DNA fragment II is subjected to electric shock, transformed into XZ-A27 bearing pKD46 plasmid, and cultivated with a LB medium with sucroses but without sodium chloride; and screening strain which has amplification product of 4210 bp identified with the primer pair consisted of a DNA fragment having a sequence of SEQ ID NO: 3 and a DNA fragment having a sequence of SEQ ID NO: 4; and designating the strain as XZ-A28; wherein the DNA fragment II was obtained with the following method: genomic DNA of Bacillus subtilis 168 is amplified with primers as set forth by SEQ ID NO: 1 and SEQ ID NO: 2 to give fragments of the alanine racemase gene, which are then inserted between the XbaI and SalI restriction sites of a pTrc99A plasmid, giving a plasmid, pTrc99A-alaR; a product amplified from pXZ-A19 as a template with the DNA fragments as set forth by SEQ ID NO: 5 and SEQ ID NO: 6 as primers is linked to the lad gene and trc promoter as well as alanine racemase gene alaR amplified from the plasmid pTrc99A-alaR with amplification sequences, such as a primer pair as set forth by SEQ ID NO: 9 and SEQ ID NO: 10, to give a plasmid, pXZ-A21, and then, plasmid DNA of the pXZ-A21 is amplified with the DNA fragments as set forth by SEQ ID NO: 3 and SEQ ID NO: 4 as primers, generating the DNA fragment II;
3) constructing a DNA fragment III, which is subjected to electric shock, transformed into XZ-A28 bearing the pKD46 plasmid, and identified with a pair of primers, consisted of a DNA fragment having a sequence of SEQ ID NO: 11 and a DNA fragment having a sequence of SEQ ID NO: 13, to give a strain with an amplification product being 1890 bp, i.e., the DL-alanine-producing engineering bacterium; the DNA fragment III has a sequence as set forth by SEQ ID NO. 16 in the sequence listing.

7. The engineering bacterium according to claim 6, characterized in that: said DL-alanine-producing engineering bacterium is Escherichia coli XZ-A30, deposited in China General Microbiological Culture Collection Center with the accession number of CGMCC No. 6667.

8. Use of the engineering bacterium of claim 1 in producing DL-alanine.

9. A method of producing DL-alanine, comprising fermenting and cultivating the engineering bacterium according to claim 1 under anaerobic or aerobic cultivation conditions, at a cultivation temperature of 30˜42° C., pH of 6.5˜7.5, and isolating and extracting the DL-alanine.

10. The method according to claim 9, characterized in that: the medium for fermenting and cultivating is consisted of raw material saccharides, nitrogen sources and trace inorganic salts, wherein, the raw material saccharides are selected from one or any combination of two or more of glucose, sucrose, fructose, xylose, maltose, lactose, galactose, Manihot esculenta, Zea mays, Beta vulgaris, lignocellulose or hydrolysate and syrup thereof; the nitrogen sources are nitrogen-containing compounds selected from one or any combination of two or more of ammonium chloride, ammonium acetate, ammonium sulfate and ammonium phosphate; and the trace inorganic salts are selected from one or any combination of two or more of soluble iron salts, cobalt salts, copper salts, zinc salts, manganese salts and molybdate.

11. The method according to claim 10, characterized in that: said medium is preferably consisted of glucose, 120 g/L; ammonium chloride, 4 g/L; NaH₂PO₄, 5 g/L; Na₂HPO₄, 5 g/L; MgSO₄.7H2O, 1 g/L; CaCl₂.2H₂O, 0.1 g/L; and trace inorganic salts, 4 ml/L; wherein, the trace inorganic salts comprise 1.5 mg of FeCl₃.6H₂O, 0.1 mg of CoCl₂.6H₂O, 0.1 mg of CuCl₂·H₂O, 0.1 mg of ZnCl₂, 0.1 mg of Na₂MoO₄.2H₂O, and 0.2 mg of MnCl₂.4H₂O₂, which are diluted with distilled water to volume of 1 L, and filtered for sterilization.

12. The method of claim 9, characterized in that: said fermenting and cultivating are lasted for 40-60 hours.

13. The method according to claim 12, characterized in that: further comprising, prior to said fermenting and cultivating, subjecting said engineering bacterium to seed cultivation for 18 hours, at a temperature of 30° C., and a rotational speed of shaker of 50 rotations/min.
